(19) United States
(12) Patent Application Publication
(10) Pub. No.: US 2005/0031715 A1
(43) Pub. Date: Feb. 10, 2005

(54) Natural preservative comprising heated garlic extract and method for preparing thereof

(75) Inventor: Kyu-Hang Kyung, Songpa-gu (KR)

Correspondence Address: FOLEY AND LARDNER;SUITE 500, 3000 K STREET NW, WASHINGTON, DC, 20007, US

(73) Assignee: SEJONG UNIVERSITY

(21) Appl. No.: 10/651,996

(22) Filed: Sep. 2, 2003

Related U.S. Application Data
(60) Provisional application No. 60/457,587, filed on Mar. 27, 2003.

(51) Int. Cl. A61K 35/78

(52) U.S. Cl. 424/754

(57) ABSTRACT

The present invention relates to a natural preservative comprising heated garlic extract and a method for preparing thereof. More particularly, the invention relates to a method for preparing a natural preservative, comprising a) inactivating alliinase contained in garlic to obtain alliinase-inactivated garlic; b) extracting said alliinase-inactivated garlic to obtain garlic extract; and c) heating said garlic extract, and a natural preservative prepared by said method. The heated garlic extract of the invention has a high antimicrobial activity, a high stability over a wide range of pH and a high storage stability without unique unpleasant flavor and taste of garlic, and has not an adverse effect of chemical preservatives on a human body. Therefore, the heated garlic extract of the invention can be effectively used as a preservative for a wide variety of products.

### TECHNICAL FIELD OF THE INVENTION

The present invention relates to a natural preservative comprising heated garlic extract and a method for preparing thereof. More particularly, the invention relates to a method for preparing a natural preservative, comprising a) inactivating alliinase contained in garlic to obtain alliinase-inactivated garlic; b) extracting said alliinase-inactivated garlic to obtain garlic extract; and c) heating said garlic extract, and a natural preservative prepared by said method.

### BACKGROUND OF THE INVENTION

A preservative generally refers to a material, when added into materials having a direct effect on human body such as medicine, foods or cosmetics and the like, preventing spoilage of the materials which is caused by microbial growth. Although the preservative can be extracted from a natural material, it is usually a synthetic material which can be easily produced in a mass amount with low cost.

The chemically synthesized preservative commonly used in food includes propionic acid, benzoic acid, sorbic acid and their salts. However, said preservatives have problems that they are effective or good enough only under the acidic condition. For example, it is known that the preservative effect of benzoic acid in pH 3.0 solution is 100 times higher than that in pH 7.0 solution. In addition, said chemical preservatives may be absorbed and accumulated inside a human body to induce a toxic effect over a long term. Moreover, some of the chemical preservatives can be carcinogenic or capable of reducing or destructing cellular functions by damaging cell membrane, reacting with cytoplasm, destructing enzyme function and degenerating proteins and the like, thus consequently weakening the immune system of a human body. For such reasons, chemical preservatives are generally avoided by a consumer. However, without an appropriate material to substitute, a consumer is left with no choice but to use them at the present moment.

Accordingly, in order to solve the above problems and satisfy the consumer's interest, studies on preservatives utilizing natural materials have been actively carried out in recent days. The Korean patent application No. 10-2000-6977 discloses a method for preparing natural preservatives using oriental herbs. The Korean patent application No. 10-2000-14283 discloses a natural preservative characterized in that it comprises a berchemin glycoside extracted from Berchemia berchemiaefolia. In addition, the Korean patent application No. 10-2001-28618 discloses a natural preservative comprising an extract of Caesalpinia sappan.

Meanwhile, a garlic (Allium sativum) is a perennial plant which belongs to Liliaceae, and is known to help blood circulation in a human body by increasing the number of erythrocytes, and to prevent adult diseases such as cancer, heart disease, or cerebral vascular disease by preventing cellular aging and enhancing body strength. It is also known that garlic is effective for enhancing body stamina and stimulating hormone secretion, and also has a strong antimicrobial effect.

The antimicrobial effect of garlic is due to allicin (allyl 2-propenethiosulfinate), which is produced by the degradation of alliin (S-allyl-L-cysteine sulfoxide) by an enzyme called alliinase (Small et al., J. Amer. Chem. Soc., 69:1710-1713, 1947). Allicin is known to be a compound which is responsible for unpleasant flavor and taste intrinsic to a fresh garlic.

There have been studies to prepare a natural preservative using the antimicrobial activity of garlic as described above. That is, the Chinese patent No. 1068714 discloses a plant preservative prepared by extracting a garlic oil from garlic bulb and distilling the garlic oil at low temperature and low pressure. The U.S. Pat. No. 5,453,420 discloses a food preservative extracted from garlic with ethanol. The Japanese patent No. 2000-4778 discloses a natural preservative for fish food, which comprises essence extracted from garlic as an ingredient of preservative solution.

However, said preservatives have a disadvantage that their preservative effect is maintained for a very short time, because fresh garlic is used as an ingredient of the preservatives. That is, since allicin which is a effective component of the antimicrobial activity is easily degraded into other compounds, the storage stability of food in which said preservatives are used is not so good (Deak and Beuchat, Handbook of food spoilage yeasts, 29-95, 1996). Further, the use of said preservatives is limited to a few types of food, due to the unpleasant taste and flavor of fresh garlic. Thus, foods in which said preservatives can be employed are limited to those with strong intrinsic taste and flavor that can mask those of garlic, such as soy sauce, fish sauce, marinated fish, meat products, mayonnaise and salad dressing that is preserved by vinegar and salt.

In the course of studying to develop a natural preservative which can substitute the synthetic chemical preservative, the inventors found that a heated garlic extract of which alliinase is inactivated has a high antimicrobial activity, a high stability over a wide range of pH and a high storage stability while unique unpleasant flavor and taste of the garlic are removed, whereby the present invention has been completed.

### BRIEF SUMMARY OF THE INVENTION

The present invention provides a method for preparing a natural preservative, comprising a) inactivating alliinase contained in garlic to obtain alliinase-inactivated garlic; b) extracting said alliinase-inactivated garlic to obtain garlic extract; and c) heating said garlic extract.

In addition, the present invention provides a natural preservative prepared by said method.

### BRIEF DESCRIPTION OF THE DRAWINGS

FIG. 1 shows the steps of the method for preparing the heated garlic extract of the invention.

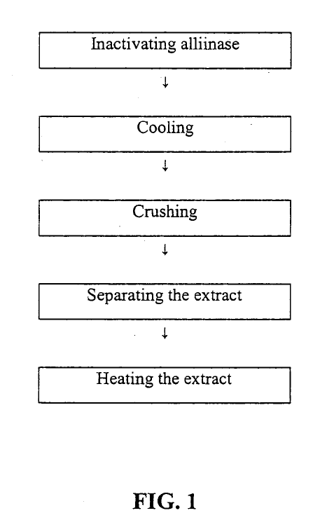

FIG. 2 shows the antimicrobial activity over the lapse of time of the garlic extract of the invention heated at 121° C., tested for S. aureus B33 and Z. rouxii KCCM 50523, respectively (◯: S. aureus B33, ▪: Z. rouxii KCCM 50523).

FIG. 3 shows the antimicrobial activity over the lapse of time of the garlic extract of the invention heated at various temperatures, tested for Candida utilis ATCC 42416.

FIG. 4 shows the antimicrobial activity over the lapse of time of the heated garlic extract of the invention, the fresh garlic extract, and the AITC (allyl isothiocyanate) solution, which have been stored at 37° C., tested for Candida utilis ATCC 42416 (Δ: heated garlic extract, ▪: fresh garlic extract, □: AITC solution).

FIG. 5 shows the antimicrobial activity of the heated garlic extract of the present invention and potassium sorbate at different pHs, tested for Candida utilis ATCC 42416 (◯: heated garlic extract, : potassium sorbate, ▪: YMPGB).

### DETAILED DESCRIPTION OF THE INVENTION

The steps of a method for preparing the heated garlic extract of the invention are described in detail below.

Step 1: Enzyme Inactivation

In order to prevent the production of allicin which is unstable and provides unpleasant taste and flavor of garlic, alliinase contained in garlic is inactivated. This inactivation step may include any method that can inactivate alliinase, such as heating; treatment with acid, alkali or salt; irradiation with UV rays or radioactive rays. In the present invention, garlic cloves were heated in hot water. The heating can be preferably carried out in hot water at 90 to 100° C. for 5 to 15 min. More preferably, it can be carried out in boiling water at 100° C. for 5 to 10 min.

Step 2: Extraction

The garlic of which alliinase is inactivated according to the step 1 is extracted with water to obtain garlic extract.

The garlic extract of this step may be prepared by crushing the alliinase-inactivated garlic cloves and subsequently separating the extract from the crushed garlic. The crushing can be achieved by using any kinds of machines or tools known to those skilled in the pertinent art. In the present invention, the alliinase-inactivated garlic cloves were crushed using a blender in which sterile water with same weight as the garlic is added. Next, in order to separate the effective component from the crushed garlic, various methods like centrifugation, cloth separation or ultrafiltration may be used. In the embodiment of the invention, the crushed garlic material was subjected to centrifugation and the supernatant was separated.

Step 3: Heating

The heated garlic extract of the invention is prepared by heating the garlic extract obtained from the step 2.

For the heating in this step, any kind of heating method including heating in a pressure cooker, autoclave, and live steam injection can be used, however, the autoclave was used in the embodiment of the invention. Heating temperature and time can vary depending on heating conditions. The garlic extract may be heated preferably, for 1 min to 10 hour at 100 to 190° C., more preferably, for 15 to 180 min at 110 to 140° C., and the most preferably, for 45 min at 121° C.

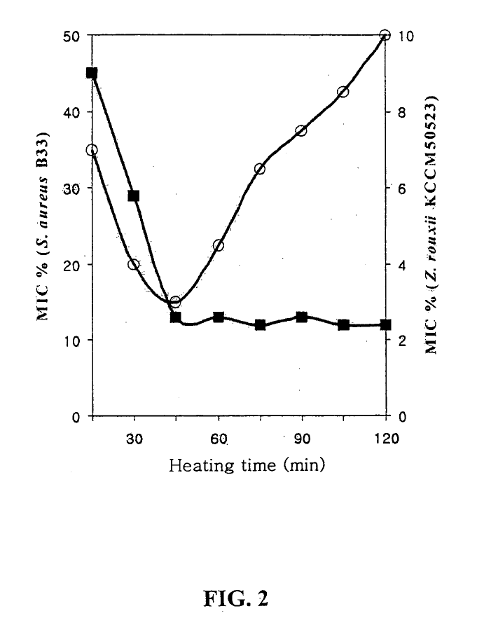

In an embodiment of the invention, the antibacterial and antifungal activity of the heated garlic extracts of the invention over the lapse of time was observed. Both of the antibacterial and antifungal activity were the highest when the garlic extract of the invention had been heated for 45 min at 121° C. However, upon prolonged heating, the antibacterial activity started to decrease while the antifungal activity remained the same with its highest activity (see FIG. 2). This result indicates that, the antibacterial activity and the antifungal activity of the heated garlic extract of the invention come from different materials, respectively, and the antifungal material is more stable under heat than the antibacterial material.

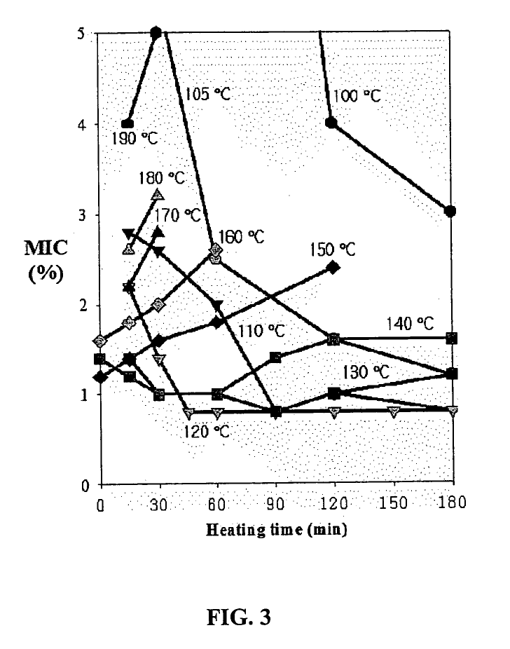

To determine the heating temperature and time at which the heated garlic extract of the invention has effective antimicrobial activity, garlic extracts heated under various conditions were prepared; i.e., heating was carried out in different time from 0 to 180 min and at different temperature from 100 to 190° C. The antimicrobial activity was measured for each of heated garlic extract above. As a result, it was found that the antimicrobial activity of the heated garlic extract is high when the garlic was heated for 1 min to 10 hour at temperature of 100 to 190° C., preferably for 15 to 180 min at temperature of 110 to 140° C., more preferably for 30 to 180 min at temperature of 120 to 130° C. and most preferably for 45 min at temperature of 121° C. (See FIG. 3). In addition, to produce a component with the antimicrobial activity at low temperature such as 100° C. or 105° C., heating for 5 to 10 hours was required (data is not shown). At high temperature such as temperature between 170° C. to 190° C., it appeared that the active component is produced by heating for 10 to 30 min but degraded soon. That is, it is believed that the time required for producing the active components depends on the heating temperature.

In another embodiment of the invention, the antimicrobial activity of the heated garlic extract of the invention was compared with that of both fresh garlic extract and AITC (allyl isothiocyanate) solution which are used as a preservative in a limited food. As a result, the antimicrobial activity of the heated garlic extract of the invention was lower than that of the fresh garlic extract and AITC solution. However, the heated garlic extract of the invention was found to have the antimicrobial activity high enough to be used as an effective natural preservative (see Table 2).

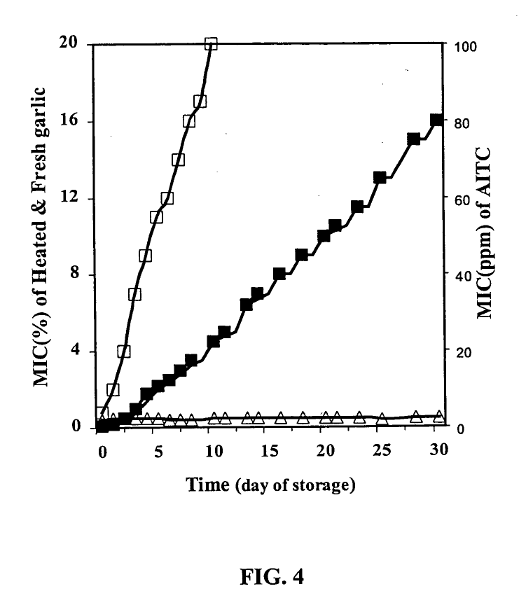

Yet in another embodiment of the invention, the storage stability (i.e., antimicrobial activity over the lapse of time) was measured and compared for the heated garlic extract of the invention, the fresh garlic extract and AITC solution. During 30 days of the test, the antimicrobial activity of the heated garlic extract of the invention remained quite stable, while those of fresh garlic extract and AITC solution were very unstable. The antimicrobial activity of the fresh garlic extract after 30 days was found more than 200 times less than its original activity (see FIG. 4). This is due to instability of allicin, an effective component of the antimicrobial activity, and its easy degradation into another chemical materials (Deak and Beuchat, Handbook of food spoilage yeasts, 29-95, 1996). In this connection, since the heated garlic extract of the invention has very good storage stability which is an essential requirement for a preservative, it can be used as an effective preservative.

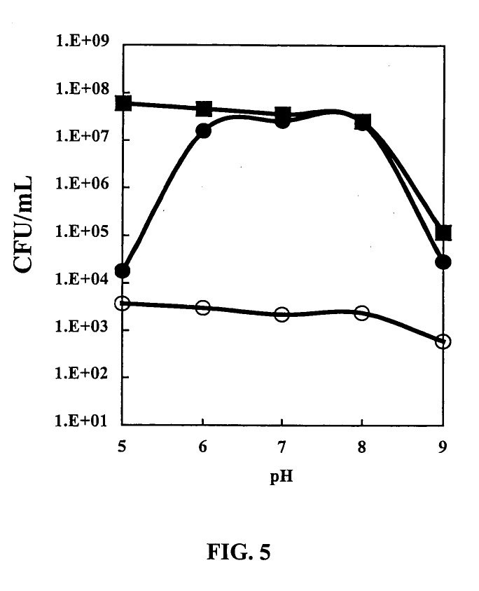

Yet in another embodiment of the invention, pH stability of the heated garlic extract of the invention was compared with that of potassium sorbate as a conventional chemical preservative. The antimicrobial activity of potassium sorbate was good only under low pH condition (i.e., acidic condition). However, the antimicrobial activity of the heated garlic extract of the invention was good in a wide range of pH including low pH condition (see FIG. 5). Accordingly, pH stability of the heated garlic extract of the invention is higher than conventional preservatives, and therefore the heated garlic extract of the invention can be used as an excellent preservative with diverse usage.

The heated garlic extract of the invention does not have unique unpleasant taste and flavor of fresh garlic. It is known that the unique unpleasant taste and flavor of fresh garlic are due to allicin that has a strong antimicrobial activity (Cavallito and Bailey, J. Amer. Chem. Soc., 66:1950-1951, 1944). The method of the invention includes the inactivation of alliinase contained in fresh garlic to inhibit formation of allicin. As such, the heated garlic extract of the invention does not have strong unpleasant taste and flavor of fresh garlic.

As described above, the heated garlic extract of the invention is free of any adverse effect present in conventional chemical preservatives and is advantageous in that its preservative effect is good over a wide range of pH. Moreover, though not as active as the fresh garlic extract or AITC solution which has been used as a natural preservative, the heated garlic extract of the invention has the antimicrobial effect to the extent that it can be effectively used as a preservative. In addition, the heated garlic extract of the invention is advantageous in that it has excellent storage stability and does not have unique unpleasant taste and flavor of fresh garlic.

Thus, the heated garlic extract of the invention can be used as an effective preservative for a wide range of commercial products. For example, such commercial products include food products such as soy sauce, bean paste, hot-bean paste and salt-preserved goods, and cosmetics and medicines, but not limited thereto.

The present invention will be described on the basis of the following Examples in more detail. However, the Examples shown below are provided solely to illustrate the invention and do not limit the invention in any way.

### EXAMPLE 1

Preparation of the Heated Garlic Extract of the Invention

Garlic (Allium sativum) used in the invention was purchased from the markets around Seoul, South Korea. Peeled and trimmed garlic cloves were heated in 100° C. water for 10 min in order to inactivate alliinase. The heated garlic was cooled down in cold water, and then put into a blender (Waring blender) containing the same amount of sterile water and crushed. After the crushing, the resultant was subjected to a centrifugation at 17,600×g for 20 min (HMR-2001V, Hanil Industrial Co., Incheon, Korea). The supernatant obtained from the centrifugation was collected in a screw-capped glass tube and heated in an autoclave under different times and temperatures as described in Table 1, respectively. After such heating, a transparent upper layer solution was obtained, whereby the garlic extracts heated at various heating temperature and time were prepared.

TABLE 1

| | 15 min | 30 min | 45 min | 60 min | 75 min | 90 min | 105 min | 120 min | 150 min | 180 min |
|---|---|---|---|---|---|---|---|---|---|---|
| 100° C. | | | | | | | | ∘ | | ∘ |
| 105° C. | | | | ∘ | | | | ∘ | | ∘ |
| 110° C. | ∘ | ∘ | | ∘ | | ∘ | | ∘ | | ∘ |
| 120° C. | ∘ | ∘ | ∘ | ∘ | | ∘ | | ∘ | ∘ | ∘ |
| 121° C. | ∘ | ∘ | ∘ | ∘ | ∘ | ∘ | ∘ | ∘ | | |
| 130° C. | ∘ | ∘ | | ∘ | | ∘ | | ∘ | | ∘ |
| 140° C. | ∘ | ∘ | | ∘ | | ∘ | | ∘ | | ∘ |
| 150° C. | ∘ | ∘ | | ∘ | | | | ∘ | | |
| 160° C. | ∘ | ∘ | | ∘ | | | | | | |
| 170° C. | ∘ | ∘ | | | | | | | | |
| 180° C. | ∘ | ∘ | | | | | | | | |
| 190° C. | ∘ | ∘ | | | | | | | | |

### COMPARATIVE EXAMPLE 1

Preparation of the Fresh Garlic Extract

Peeled and trimmed garlic cloves were put into a blender (Waring blender) containing the same amount of sterile water and crushed. After the crushing, the resultant was centrifuged at 17,600×g for 30 min (HMR-2001V, Hanil Industrial Co., Incheon, Korea), and consequently subjected to sterile filtering (0.45 μm, Gelman Sci., Ann Arbor, Mich., USA) to obtain the fresh garlic extract.

### COMPARATIVE EXAMPLE 2

Preparation of the AITC Solution

AITC compound, which is found in mustard but not in garlic, was used in the present invention for the purpose of comparison. AITC compound used in the invention was purchased from Acros Orgnics Co. AITC compound was dissolved with 0.05% Tween 80 as a surfactant in YMPGB medium to prepare 1000-ppm stock solution. The resulting solution was subjected to sterile filtering.

### COMPARATIVE EXAMPLE 3

Preparation of the Potassium Sorbate Soultion

Potassium sorbate as a conventional chemical preservative was purchased from Duksan Pure Chemicals Co., LTD. (Kyonggi-do, Korea). The potassium sorbate was added into YMPGB medium with the concentration of 0.1 volume %.

### TEST EXAMPLE 1

Comparison Antibacterial and Antifungal Activity of the Garlic Extract of the Invention Over the Lapse of Time

In order to compare the antibacterial activity and the antifungal activity of the garlic extract of Example 1 prepared by heating at 121° C., according to the heating time, MIC (minimum inhibitory concentration) was measured for the samples of the heated garlic extract. The MIC represents minimum concentration of material to inhibit the growth of certain type of microorganism. It is understood that the lower the MIC of a material, the higher the antimicrobial activity of the material. The inhibition of microbial growth was measured by viable count method based on Spiral Autoplate System of Spiral Biotech Inc. Three separate experiments were carried out for each sample and the highest value was taken as the MIC of the sample.

S. aureus B33 used in this Test Example was kindly provided from Dr. Henry P. Fleming at the Food Fermentation Laboratory of the North Carolina State University in USA, and Z. rouxii KCCM 50523 was purchased from the Korean Center for Conservation of Microorganisms (KCCM). The garlic extracts were added to TSB (tryptic soy broth) medium and YMPGB (yeast extract-malt extract-peptone-glucose broth) medium respectively, which garlic extracts had been heated at 121° C. for various times of 15 min, 30 min, 45 min, 60 min, 75 min, 90 min, 105 min and 120 min respectively. Thereafter, those mediums were inoculated with S. aureus B33 and Z. rouxii KCCM 50523, respectively, to give initial number between 10⁴˜10⁵ cells/Ml, and incubated at 30° C. for 24 hrs and 48 hrs, respectively. The MIC value was determined for each sample that has been prepared with different heating time. As a result, for S. aureus B33 the antibacterial activity of the heated garlic extract of the invention was the highest when the extract had been heated for 45 min and decreased when heated for more than 45 min. For Z. rouxii KCCM 50523, the antifungal activity of the heated garlic extract of the invention was also the highest when the extract had been heated for 45 min, however it remained almost the same when heated for more than 45 min(FIG. 2).

According to the above result, it was found that the heated garlic extract of the invention has both the antibacterial activity and the antifungal activity. Further, it was assumed that the material(s) showing the antibacterial activity is(are) different from that showing the antifungal activity, and the former is more stable to the heat than the latter.

### TEST EXAMPLE 2

Antimicrobial Activity Change of the Heated Garlic Extract of the Invention Over Various Heating Time and Temperature

The YMPGB medium containing the separate garlic extract which had been prepared by heating at different temperatures of 100, 105, 110, 120, 130, 140, 150 (5 KG/CM²), 160, 170, 180, 190° C. in Example 1 was inoculated with Candida utilis ATCC 42416 to give initial number of 2.3×10⁴ CFU/mL and subsequently incubated at 30° C. C. utilis ATCC 42416 used in this Test Example was kindly provided from Dr. Henry P. Fleming at the Food Fermentation Laboratory of the North Carolina State University in USA. The resulting medium was incubated for 48 hr at 30° C. and MIC was measured for the samples of the heated garlic extract that had been prepared with various heating time, using the viable count method described in Test Example 1.

As illustrated in FIG. 3, the activity of the garlic extract samples varied with the heating time, but it was confirmed that the material(s) having an antimicrobial activity was produced. In addition, the heating time required to produce such material(s) having the antimicrobial activity depended on the heating temperature. Especially, it was found that the antimicrobial activity of the heated garlic extract was high, which had been produced by heating for more than 90 min at 110° C., more than 45 min at 120° C., 30 to 180 min at 130° C. and 15 to 60 min at 140° C.

### TEST EXAMPLE 3

Comparison of Antimicrobial Activity for the Heated Garlic Extract of the Invention the Fresh Garlic Extract and the AITC Solution

Antimicrobial activity was measured for the garlic extract heated at 121° C. for 45 min of Example 1, the fresh garlic extract and the AITC solution which had been prepared in Comparative Examples. Conditions for the viable count method, the medium and initial number of microorganisms inoculated were the same as those described in the above Test Examples. The MIC was measured at 24 hrs or 48 hrs after incubating the bacteria or the yeasts, respectively.

Staphylococcus aureus B33, Escherichia coli B34, Enterobacter aerogenes B146, Leuconostoc mesenteroides LA10, Pediococcus pentosaceus LA3, Lactobacillus plantarum LA97, Pichia membranifaciens Y20, Saccharomyces cerevisiae ATCC 4126 and Candida utilis ATCC 42416 used in this Test Example were kindly provided from Dr. Henry P. Fleming at the Food Fermentation Laboratory of the North Carolina State University in USA. Candida albicans HY1 was separated from an infant with oral candidiasis. In addition, Candida albicans KCTC 7121 and 7965 were purchased from Korean Collection for Type Cultures (KCTC) and Zygosaccharomyces bisporus KCCM 50168, Zygosaccharomyces rouxii (soya) KCCM 11300, Zygosaccharomyces rouxii KCCM 11303, Zygosaccharomyces rouxii (sake) KCCM 50523 and Zygosaccharomyces rouxii (gracilis) KCCM 50546 were obtained from KCCM (Korean Culture Center of Microorganisms).

The MIC of the heated garlic extract of the invention was about 7 to 10 times or 1500 to 6000 times higher than that of the fresh garlic extract and the AITC solution, respectively. From that, it can be seen that the antimicrobial activity of the heated garlic extract of the invention is lower than that of the fresh garlic extract and the AITC solution. Nevertheless, the antimicrobial activity of the heated garlic extract of the invention was high enough to be used as an effective preservative. The inhibitory activity of the heated garlic extract of the invention over xerotolerant yeast (MIC: 1.5˜3.0 volume %) was lower than that over non-xerotolerant yeast (MIC: 0.5˜1.0 volume %), but it was higher than that over bacteria (Table 2).

TABLE 2

| | MIC | | |
|---|---|---|---|
| | Heated Garlic | Fresh Garlic | AITC solution |
| Microorganism | (volume %) | (volume %) | (volume %) |
| Staphylococcus aureus B33 | >20 | 1.5 ± 0.1 | 0.015 ± 0.0006 |
| Escherichia coli B34 | >20 | 1.0 ± 0 | 0.01 ± 0.0006 |
| Enterobacter aerogenes B146 | >20 | 1.5 ± 0 | 0.02 ± 0.002 |
| Leuconostoc mesenteroides LA10 | >20 | 2.0 ± 0 | 0.04 ± 0.0023 |
| Pediococcus pentosaceus LA3 | >20 | 3.0 ± 0.3 | 0.02 ± 0.0012 |
| Lactobacillus plantarum LA97 | >20 | 2.0 ± 0.1 | 0.02 ± 0 |
| Candida albicans (Clinical) | 0.8 ± 0.1 | 0.1 ± 0.01 | 0.0003 ± 0 |
| Candida albicans KCTC 7121 | 0.9 ± 0.1 | 0.075 ± 0 | 0.0003 ± 0.0001 |
| Candida albicans KCTC 7965 | 1.0 ± 0.1 | 0.1 ± 0.01 | 0.0004 ± 0.0001 |
| Candida utilis ATCC 42416 | 0.6 ± 0 | 0.075 ± 0.01 | 0.0004 ± 0.0001 |
| Saccharomyces cerevisiae ATCC | 0.7 ± 0.1 | 0.075 ± 0.01 | 0.0002 ± 0.0001 |
| 4126 | | | |
| Pichia membranefaciens Y20 | 0.5 ± 0 | 0.075 ± 0.01 | 0.0001 ± 0 |
| Zygosaccharomyces bisporus | 1.5 ± 0.1 | 0.2 ± 0.01 | 0.0002 ± 0 |
| KCCM 50168 | | | |
| Zygosaccharomyces rouxii KCCM | 2.5 ± 0.3 | 0.25 ± 0.03 | 0.0002 ± 0 |
| 11300 | | | |
| Zygosaccharomyces rouxii KCCM | 2.5 ± 0.2 | 0.30 ± 0.03 | 0.0004 ± 0.0001 |
| 11303 | | | |
| Zygosaccharomyces rouxii KCCM | 3.0 ± 0.3 | 0.25 ± 0 | 0.0005 ± 0.0001 |
| 50523 | | | |
| Zygosaccharomyces rouxii KCCM | 2.5 ± 0.2 | 0.2 ± 0.01 | 0.0004 ± 0.0001 |
| 50546 | | | |

### TEST EXAMPLE 4

Comparison of Antimicrobial Activity of the Heated Garlic Extract of the Invention, the Fresh Garlic Extract and the AITC Solution, Over Storage Time

Antimicrobial activity of the garlic extract prepared by heating at 121° C. for 120 min in Example 1, the fresh garlic extract and the AITC solution was measured for Candida utilis ATCC 42416, over storage time. Candida utilis ATCC 42416 used in this Test Example was kindly provided from Dr. Henry P. Fleming at the Food Fermentation Laboratory of the North Carolina State University in USA. The heated garlic extract and the fresh garlic extract were added to the YMPGB, respectively, and Candida utilis ATCC 42416 was inoculated with an initial number of 1.9-2.7×10⁴ CFU/mL and subsequently stored at 37° C. From the first day to the 30^{th} day of the storage, the MIC was measured everyday or every two days using the viable count method described in Test Example 1.

The antifungal activity of the heated garlic extract of the invention was very stable during the whole test period of 30 days. However, the antifungal activities of the fresh garlic extract and the AITC solution were very unstable and the MIC of the fresh garlic extract jumped about 200 times, i.e., from the initial value of 0.075 volume % to 16.000 volume % (FIG. 4).

### TEST EXAMPLE 5

Comparison of Antimicrobial Activity of the Heated Garlic Extract of the Invention and Potassium Sorbate, Over pH Condition

Antimicrobial activity of the garlic extract heated at 121° C. for 120 min in Example 1 was measured for Candida utilis ATCC 42416, under various pH conditions. The same measurement was carried out for potassium sorbate as a conventional chemical preservative. For a control, YMPGB medium without comprising any preservative was used. Candida utilis ATCC 42416 used in this Test Example was kindly provided from Dr. Henry P. Fleming at the Food Fermentation Laboratory of the North Carolina State University in USA. After adjusting pH of the YMPGB medium to pH 4, 5, 6, 7, 8 or 9 by adding 1N HCl or 1N NaOH solution, the heated garlic extract in 0.5 volume % and potassium sorbate in 0.1 weight % were added into the YMPGB medium, respectively. Then, Candida utilis ATCC 42416 was inoculated with an initial number of 2.1-2.3×10⁵ CFU/mL and subsequently incubated at 30° C. for 24 hrs. After completing the incubation, the value of log CFU/Ml was measured by using the viable count method described above.

As illustrated in FIG. 5, the antimicrobial activity of potassium sorbate was significantly affected by pH. However, the antimicrobial activity of the heated garlic extract of the invention stayed high without depending on pH. The antimicrobial activity of potassium sorbate was high at pH 5, but at pH 6, it dropped to almost zero as same as the control group of the YMPGB medium only. This is due to the fact that only the undissociated form of sorbic acid has the antimicrobial activity and such activity is lost at high pH where sorbic acid dissociates (Macris B J, Appl. Microbiol., 30(4); 503-506, 1975).

### TEST EXAMPLE 6

Measurement of the Preservative Effect of the Heated Garlic Extract of the Invention Added to Soy Sauce

It was determined that the heated garlic extract of the invention can effectively inhibit the film formation on surface of soy sauce, which is caused by soy sauce film yeast. Soy sauce film yeast, Zygosaccharomyces rouxii SS1 was obtained from a local soy sauce maker, Haechandeul Food Co. (Kongju, Korea). Fresh soy sauce without comprising any preservative was obtained from another soy sauce marker, Daesang Food Co. (Suncheon, Korea).

The garlic extract of the invention which had been produced by heating at 121° C. for 45 min was added to the soy sauce clarified by the sterile filtering in a volume of 0, 0.5, 1.0, 1.5, 2.0 or 2.5 volume %, respectively. Then, Z. rouxii SS1 was inoculated with an initial number of 1.5˜2.3×10⁵ CFU/Ml. The inoculated soy sauce was incubated at 30° C. for 30 days while the formation of film on the surface of the soy sauce was checked everyday.

As illustrated in Table 3, the heated garlic extract of the invention effectively inhibited the film formation by soy sauce film yeast on the surface of soy sauce. Film was formed just after 5 days on the surface of soy sauce not treated with the heated garlic extract. However, for the soy sauce treated with the heated garlic extract, day of film formation was delayed as the concentration of the garlic extract increased. For the soy sauce treated with the heated garlic extract with more than 2.0 volume %, no surface film was formed during the entire 30 days of experiment (see Table 3).

TABLE 3

| Heated garlic | Day of film |
|---|---|
| (volume %) | appearance |
| 0 | 5 |
| 0.5 | 6 |
| 1.0 | 7 |
| 1.5 | 9 |
| 2.0 | >30 |
| 2.5 | >30 |

As described the above, the heated garlic extract of the invention has a high antimicrobial activity, a high stability over a wide range of pH and a high storage stability without unique unpleasant flavor and taste of garlic, and has not an adverse effect of chemical preservatives on a human body. Therefore, the heated garlic extract of the invention can be effectively used as a preservative for a wide variety of products.

1. A method for preparing a natural preservative, comprising:
a) inactivating alliinase contained in garlic to obtain alliinase-inactivated garlic;
b) extracting said alliinase-inactivated garlic to obtain garlic extract; and
c) heating said garlic extract.

2. The method according to claim 1, wherein said step a) is heating garlic in water at 90° C. to 100° C. for 5 min to 15 min.

3. The method according to claim 1, wherein said step b) is crushing and centrifuging said alliinase-inactivated garlic to obtain garlic extract.

4. The method according to claim 1, wherein said step c) is heating said garlic extract at 100° C. to 190° C. for 10 min to 10 hr.

5. The method according to claim 1, wherein said step c) is heating said garlic extract at 110° C. to 140° C. for 15 min to 180 min.

6. The method according to claim 1, wherein said step c) is heating said garlic extract at 121° C. for 45 min.

7. A natural preservative which is prepared by the method according to any one of claims 1 to 6.
